(19) United States
(12) Patent Application Publication
(10) Pub. No.: US 2026/0308566 A1
(43) Pub. Date: Oct. 8, 2026

(54) AUTOMATED EXTRACTION OF NUCLEIC ACID SAMPLES FROM A PLURALITY OF SAMPLES

(71) Applicant: UNTAP LTD, London (GB)

(72) Inventors: Navid ASLANI, London (GB); Jay BULLEN, London (GB)

(73) Assignee: UNTAP LTD, London (GB)

(21) Appl. No.: 19/482,307

(22) PCT Filed: May 10, 2024
(86) PCT No.: PCT/EP2024/062942

§ 371 Date: Nov. 7, 2025

(30) Foreign Application Priority Data
May 10, 2023 (GB) 2306923.0
Sep. 25, 2023 (GB) 2314626.9

(51) Int. Cl. C12N 15/10; B01L 3/00

(52) U.S. Cl. CPC C12N 15/1003; B01L 3/502; B01L 2200/028; B01L 2200/0631; B01L 2200/16; B01L 2300/0832; B01L 2300/0861; B01L 2400/0433

(57) ABSTRACT

The present invention provides devices useful for automated extraction of nucleic acid samples from a plurality of samples. The present invention further provides processes for automated extraction of nucleic acid samples from a plurality of samples, which can be carried out using devices of the invention.

### FIELD

The present disclosure relates to devices and methods for automated extraction of nucleic acid samples.

### BACKGROUND

Polymerase chain-reaction (PCR)-based techniques, such as quantitative PCR (qPCR), or reverse transcription qPCR (RT-qPCR) enable highly accurate and sensitive detection of nucleic acids in samples of interest. During the COVID-19 pandemic it was demonstrated that even though PCR-based techniques can be employed rapidly to enable testing of large numbers of samples, the current state of the art does not allow for automation of the process of nucleic acid extraction without incurring considerable cost, which can result inter alia from use of disposable components, wherein the matrix used for extraction of nucleic needs to be disposed of and/or exchanged following a single nucleic acid extraction. Additionally, the use of single-use disposable matrices for nucleic acid extraction increases the overall footprint of the device, by creating the need to accommodate a large plurality of receptacles housing the matrices for nucleic acid to allow for a limited number of extraction cycles.

Moreover, current devices for automated extraction of nucleic acids are unable to operate continuously in remote and/or inaccessible locations. For instance, robot pipettor instruments with a design similar to a 3D printer are prone to malfunction due to the 3 degrees of freedom in which the pipettor needs to move and significant distances travelled by the moving parts (which is also linked to a large footprint). Pipette tips used in the robot pipettors are also predominantly designed to be disposable and hence are prove difficult to clean and/or decontaminate between samples, further increasing the need for waste disposal in robot pipettor-based instruments. It should also be recognized that for example, the volumes of the samples and or reagents provided during the extraction of nucleic acids by the devices for automated extraction of nucleic acids will change with wear and tear of the components involved. Pre-packaged and pre-measured reagents provide a non-cost-effective alternative, which additionally creates an additional need for the device to comprise means for waste-management, further increasing the cost and footprint of such a device, while also increasing the engineering complexity of the device by introducing the need to provide means for releasing the reagents from the packaging. This further limits the utility of the devices for automated extraction of nucleic acids known in the art, e.g., for continuous monitoring of biomarkers in environmental samples. In theory, a skilled person could consider the use of precise liquid handlers e.g. acoustic-based liquid handling (such as an Echo machine) to decrease the effect of wear and tear on the accuracy and precision of volume measurements. However, due to their mode of operation, acoustic liquid handlers are unlikely to be suitable for applications such as for example the extraction of nucleic acids for environmental monitoring of pathogens, due to the small orders of volumes of the liquids used in acoustic liquid handling. For acoustic-based handling, the receptacle receiving the liquid transferred is usually placed upside down, meaning liquid volumes in the millilitre ranges would likely leak back down from the receptacle. Additionally, acoustic liquid handlers are characterised by a considerable size and hardware cost and thus their potential use in the devices for automated extraction of nucleic acid is not desirable.

Therefore, the need exists to provide improved devices and methods for automated extraction of nucleic acid samples from a plurality of samples.

### SUMMARY OF THE INVENTION

Accordingly, it is an object of the present invention to provide improved devices and processes for automated extraction of nucleic acid samples from a plurality of samples.

The invention provides the following aspects.

### I. Aspect I—Sensor-Focused Device

In a first aspect the invention provides a device for automated extraction of nucleic acid samples from a plurality of samples, comprising:
i) a sample module comprising:
   a) a plurality of receptacles for receiving the plurality of samples, wherein each receptacle of the plurality of receptacles comprises a matrix for nucleic acid binding,
   b) means for housing the plurality of receptacles, and
   c) an extraction unit configured to extract the nucleic acid samples from any sample of the plurality of samples; and
ii) a multi-channel pump module, comprising:
   d) a plurality of reservoirs for storing reagents for extraction of nucleic acid samples, and
   e) at least one pump configured to deliver the plurality of samples and/or the reagents for extraction of nucleic acid samples to and/or through the matrix of any receptacle of the plurality of receptacles; and wherein:
      the device comprises means for measuring the volume of analyte in any receptacle of the plurality of receptacles,
      the multi-channel pump module and the sample module are connected such that the reagents can be delivered to and/or through any matrix of the plurality of receptacles to enable the extraction of the nucleic acid samples from the plurality of samples, and
      the means for housing the plurality of receptacles is configured such that any of the plurality of the receptacles can be positioned for delivery of the reagents from the extraction unit.

### II. Aspect II—Sensor-Focused Process

In a second aspect the invention provides a process for automated, matrix-based extraction of nucleic acid samples from a plurality of samples, comprising the steps of:
a) receiving a sample of the plurality of samples in a receptacle of a plurality of receptacles,
b) binding a nucleic acid to a matrix,
c) washing the matrix, and
d) eluting the nucleic acid from the matrix;
   wherein a volume of sample used in the process and/or a volume of any reagent of a plurality of reagents used in the process are controlled using image processing, and
   wherein image processing is used to measure the volume of the sample and/or the volume of any reagent of a plurality of reagents received in the receptacle.

### III. Aspect III—Regeneration-Focused Device

In a third aspect the invention provides a device for automated extraction of nucleic acid samples from a plurality of samples, comprising:
i) a sample module comprising:
   a) a plurality of receptacles for receiving the plurality of samples, wherein each receptacle of the plurality of receptacles comprises a matrix for nucleic acid binding,
   b) means for housing the plurality of receptacles,
   c) an extraction unit configured to extract the nucleic acid samples from any sample of the plurality of samples, and
   d) a regeneration unit configured to regenerate the matrix in any receptacle of the plurality of receptacles; and
ii) a multi-channel pump module, comprising:
   e) a plurality of reservoirs for storing reagents for extraction of nucleic acid samples and a plurality of reservoirs for storing reagents for regeneration of the matrix, and
   f) at least one pump configured to deliver the plurality of samples, the reagents for extraction of nucleic acid samples and/or the reagents for regeneration of the matrix to and/or through the matrix of any receptacle of the plurality of receptacles; and wherein:
      the multi-channel pump module and the sample module are connected such that the reagents can be delivered to and/or through any matrix of the plurality of receptacles to enable the extraction of the nucleic acid samples from the plurality of samples and such that any matrix of the plurality of receptacles can be regenerated, and
      the means for housing the plurality of receptacles is configured such that any of the plurality of the receptacles can be positioned for delivery of the reagents from the extraction unit or the regeneration unit.

### IV. Aspect IV—Regeneration-Focused Process

In a fourth aspect the invention provides a process for automated, matrix-based extraction of nucleic acid samples from a plurality of samples using a plurality of matrices for nucleic acid binding, the process comprising a nucleic acid extraction step and a matrix regeneration step, wherein the nucleic acid extraction step and the matrix regeneration step are performed in-parallel.

### BRIEF DESCRIPTION OF THE DRAWINGS

Select embodiments of the disclosed technology will now be described in more detail with reference to the following figures:

FIG. 1: A process diagram for nucleic acid extraction using image processing to provide reproducible reagent volumes measured directly within the receptacle containing the membrane. The diagram demonstrates an outline of exemplary the steps performed during the process of aspects of the invention.

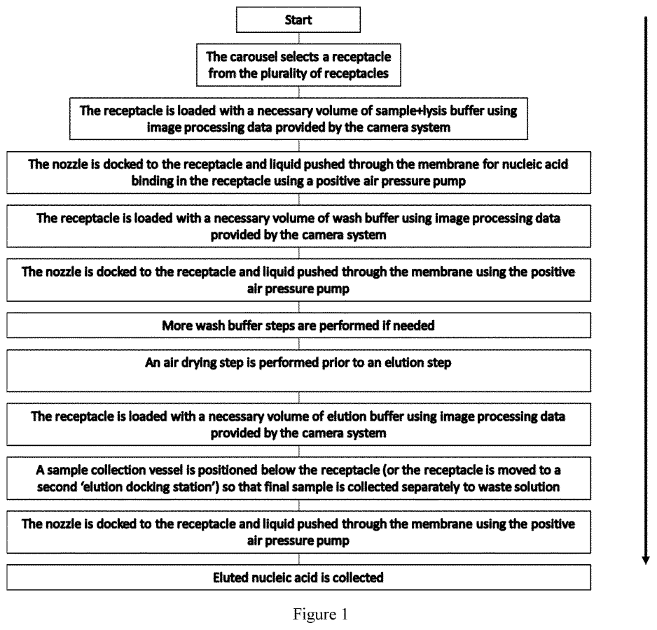

FIG. 2: A logic diagram for achieving reproducible, accurate and precise measurement of reagent volumes using image processing. The diagram demonstrates an example of the feedback loop used in aspects of the invention.

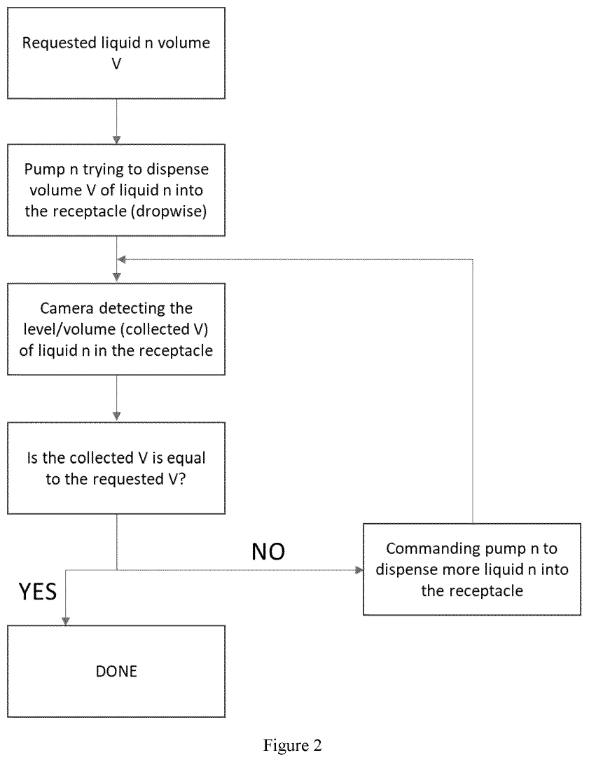

FIG. 3: An overview of the camera system and image processing employed in the devices and processes of the invention. FIG. 3A shows the arrangement of components for the application of refraction techniques to improve contrast in the application of image processing for the measurement of reagent volumes within the receptacle that houses the membrane for nucleic acid extraction. In the preferred embodiment, a plurality of receptacles are housed on a carousel. The camera either has a wide field of vision to analyse multiple receptacles, or the receptacles are brought to the camera's field of vision via a carousel or by other mechanical means. FIG. 3B shows the camera-image plane of the refraction-based liquid level detector, demonstrating the application of refraction techniques to improve the contrast when using image processing with a camera to measure reagent volumes within the receptacle used for nucleic acid extraction. The liquid level is determined in the refracted image area. Pixel brightness is measured in the area marked as “refracted image” in FIG. 3B.

FIG. 4: Exemplary test data showing a single receptacle with the membrane used 10 times to extract nucleic acids from positive and negative spiked samples. The process was entirely automated. A regeneration step consisting of a bleach wash followed by water rinse was performed between each nucleic acid extraction. The negative samples were used to evaluate residual nucleic acid (i.e. cross-contamination): the 2.5 log reduction in gene copy numbers (gene units per mL) indicates >99% removal of nucleic acid from the receptacle's membrane. The input sample was 1 mL wastewater spiked with 50 uL of a SARS-CoV-2 RNA standard (enveloped in viral capsids, NIBSC). The lysis buffer, wash buffers and elution buffers were from a typical commercial kit. The extracted nucleic acids were quantified using a commercial RT-qPCR assay on a commercial qPCR thermocycler instrument with an external calibration curve and included positive controls.

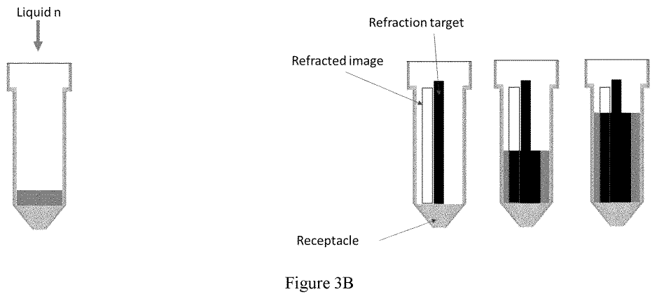

FIG. 5: Exemplary nucleic acid extraction data collected using three membranes loaded simultaneously onto the carousel test data. The data shows multiple receptacles loaded onto the carousel of the nucleic acid extraction module simultaneously and each receptacle re-used multiple times to extract nucleic acids from positive and negative spiked samples. The process was entirely automated. A regeneration step consisting of a bleach wash followed by water rinse was performed between each nucleic acid extraction. The negative samples were used to evaluate residual nucleic acid (i.e. cross-contamination): the 2-3 log reduction in gene copy numbers (gene units per mL) indicates up to 99.9% removal of nucleic acid from the receptacle's membrane. The input sample was 1 mL wastewater spiked with 50 uL of a SARS-CoV-2 RNA standard (enveloped in viral capsids, NIBSC). The lysis buffer, wash buffers and elution buffers were from a typical commercial kit. The extracted nucleic acids were quantified using a commercial RT-qPCR assay on a commercial qPCR thermocycler instrument with an external calibration curve and included positive controls.

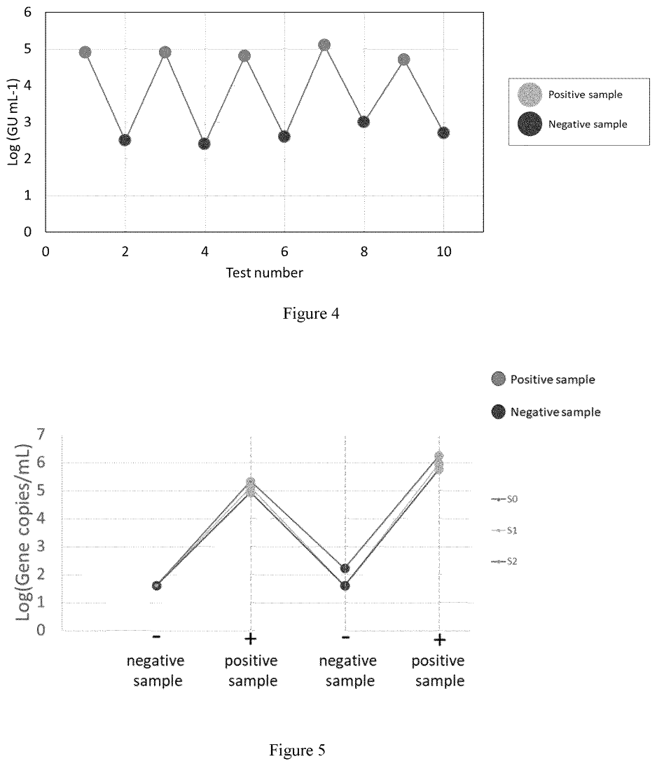

FIG. 6: An exemplary embodiment of the system of the invention wherein the at least one pump is a syringe pump in communication with a valve terminal. The communication depicted in FIG. 6 between the single pump and the valve terminal, the reagent reservoirs, the extraction unit, the regeneration unit and the in-line sample lysis module is configured to enable positive (air) pressure to be provided from the single pump and through the valve terminal. The communication depicted in FIG. 6 between the reagent reservoirs for storing reagents for nucleic acid extraction and the projections (e.g. pipette tips), between the regeneration reagent reservoirs, the in-line sample lysis module and the projections (e.g. pipette tips) is configured to enable the delivery of the reagents and sample to the receptacle.

FIG. 7: An exemplary embodiment of the system of the invention wherein the at least one pump comprises a syringe pump in communication with a valve terminal and a plurality of metering pumps. The communication between the single pump, the valve terminal and the extraction and regeneration units and the in-line sample lysis module is configured to enable positive (air) pressure to be provided from the single pump and through the valve terminal. The communication between the reagent reservoirs for storing nucleic acid extraction reagents, the metering pumps and the projections (e.g. pipette tips); between the regeneration reagent reservoirs, the metering pumps and, the in-line sample lysis module and the projections (e.g. pipette tips) is configured to enable the delivery of the reagents and sample to the receptacle.

### DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of contemplated embodiments described herein.

Any part of this disclosure may be read in combination with any other part of the disclosure, unless otherwise apparent from the context.

References to subject-matter disclosed or described “herein” relates to subject-matter disclosed or described anywhere in the present application.

The singular terms “a,” “an,” and “the” include plural referents unless context clearly indicates otherwise. Similarly, the word “or” is intended to include “and” unless the context clearly indicates otherwise.

As used herein the term “analyte” is used to describe any liquid and/or suspension, such as a sample and/or a reagent.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.”

In the specification and claims, the term “about” is used to modify, for example, the quantity of an ingredient in a composition, concentration, volume, process temperature, process time, yield, flow rate, pressure, and like values, and ranges thereof, employed in describing the examples of the disclosure. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture and amounts that differ due to mixing or processing a formulation with a particular initial concentration or mixture. Where modified by the term “about” the claims appended hereto include equivalents to these quantities.

As used herein the term “comprising” or “comprises” is used with reference to devices for automated extraction of nucleic acids, uses, compositions, methods, and respective component(s) thereof, that are essential to the method or composition, yet open to the inclusion of unspecified elements, whether essential or not.

As used herein the term “matrix” or “matrix for nucleic acid binding” is used with reference to any matrix suitable for binding of nucleic acid binding. Suitable matrices are known in the art and include e.g., silica matrices (such as silica membranes), zeolite or glass fibre matrices. Paper (or cellulose-based) matrices are also known in the art.

As used herein, the term “nozzle” refers to an apparatus for docking onto a receptacle for supplying positive air pressure to the receptacle. For the avoidance of doubt, in the context of the present disclosure the term “nozzle” is not to be interpreted as limited a cylindrical or round spout at the end of a pipe, hose, or tube used to control a jet of gas or liquid.

As used herein, the term “polynucleotide,” “nucleotide,” nucleic acid” “nucleic acid molecule” and other similar terms are used interchangeable and include DNA, RNA, mRNA and the like.

As used herein the term “reagent” is used to describe any substance which is used in a process such as nucleic acid extraction and/or matrix regeneration. The skilled person will recognize that as used herein the term “reagent” includes lysis buffers, wash buffers, elution buffers, water, hypochlorites (such as sodium hypochlorite also commonly referred to as “bleach”) and any other substances known in the art to be used in the processes of nucleic acid extraction and/or matrix regeneration. Reagents for nucleic acid extraction are readily available from commercial vendors and a skilled person will recognize that a plurality of such reagents from commercially available kits could be comprised by the devices and methods of the invention. An example of a kit comprising reagents for nucleic acid extraction is Qiagen Spin Miniprep kit.

As used herein the term “matrix regeneration” refers to the process of washing, cleaning or otherwise regenerating a matrix that has been used in a nucleic acid extraction process such that it can be used again for further nucleic acid binding according the processes disclosed herein. Regeneration solutions (also referred to herein as “cleaning solutions”) for regenerating the matrix may comprise reagents such as bleach (referred to herein as a chemical regeneration reagent), water, phosphate buffer solution (PBS) or borate buffers.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present disclosure was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol.152, S. L. Berger and A. R. Kimmel Eds., Academic Press Inc., San Diego, USA (1987); Current Protocols in Protein Science (CPPS) (John E. Coligan, et al., ed., John Wiley and Sons, Inc.), Current Protocols in Cell Biology (CPCB) (Juan S. Bonifacino et al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various examples of the disclosure.

### DETAILED DESCRIPTION OF THE INVENTION

In the first aspect of the invention, a device for automated extraction of nucleic acid samples from a plurality of samples comprises:
i) a sample module comprising:
   a) a plurality of receptacles for receiving the plurality of samples, wherein each receptacle of the plurality of receptacles comprises a matrix for nucleic acid binding,
   b) means for housing the plurality of receptacles, and
   c) an extraction unit configured to extract the nucleic acid samples from any sample of the plurality of samples; and
ii) a multi-channel pump module, comprising:
   d) a plurality of reservoirs for storing reagents for extraction of nucleic acid samples, and
   e) at least one pump configured to deliver the plurality of samples and/or the reagents for extraction of nucleic acid samples to and/or through the matrix of any receptacle of the plurality of receptacles; and wherein:
      the device comprises means for measuring a volume of an analyte in any receptacle of the plurality of receptacles,
      the multi-channel pump module and the sample module are connected such that the reagents can be delivered to and/or through any matrix of the plurality of receptacles to enable the extraction of the nucleic acid samples from the plurality of samples, and
      the means for housing the plurality of receptacles is configured such that any of the plurality of the receptacles can be positioned for delivery of the reagents from the extraction unit.

In some embodiments, the at least one pump is a single pump. In some embodiments, the at least one pump is a plurality of pumps.

In some embodiments, the extraction unit comprises a plurality of tubes configured to deliver the sample and/or the reagents to the matrix.

In some embodiments, the volume measurement provided by the means for measuring the volume of the analyte is used to control the at least one pump. In particularly preferred embodiments, the volume measurement is used to control the at least one pump using a closed loop feedback system. In some particularly preferred embodiments, the control of the at least one pump is performed continuously. The control of the plurality of the pumps using the volume measurement allows for the adjustment of pump activity to deliver an accurate and precise volume of the sample and/or reagent, without the need for servicing the device and manually recalibrating the pumps. In some embodiments, the control of the plurality of the pumps using the volume measurement allows for the adjustment of pump activity to deliver the sample and/or reagent through the matrix. The skilled person will recognize that the volume of the sample and/or reagent delivered by the at least one pump, with pump control parameters fixed and unadjusted during the operation of the device, would be expected to change over time due to wear and tear of the components of the device, resulting in a deterioration of both accuracy and precision of the device. Measuring the volume directly in the receptacle overcomes this problem. Additionally, the measurement of volumes directly in the receptacle allows to overcome the issues caused by the “dead-volumes” resulting from the volume of the tubing and other comprised by the device, which would have to be carefully considered and accounted for, should the volume measurement in the receptacle not be used. Moreover, the expansion and compression of air additionally creates a “non-linearity” of pressure differences in the syringe pump, further limiting the accuracy and precision when no volume measurements in the receptacle are performed.

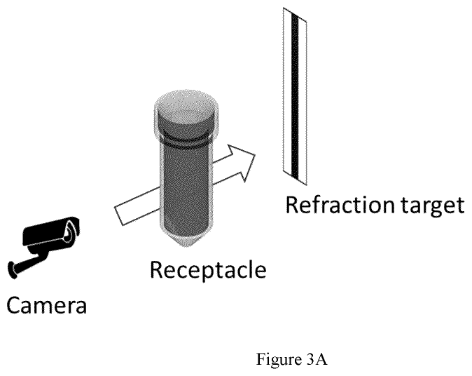

The skilled person will recognize that a plurality of embodiments can be employed to provide means for measuring the volume of analyte in any receptacle of the plurality of receptacles. In some embodiments, the means for measuring the volume of analyte in any receptacle of the plurality of receptacles comprises a weight-measurement system. In some embodiments, the means for measuring the volume of analyte in any receptacle of the plurality of receptacles comprises a load cell. In some embodiments, the means for measuring the volume of analyte in any receptacle of the plurality of receptacles comprises a sound-based system, optionally an ultrasound-based system. In preferred embodiments, the means for measuring the volume of analyte in any receptacle of the plurality of receptacles comprises a camera system. In preferred embodiments of the invention the camera system can be positioned in sufficiently close proximity to the plurality of tubes configured to deliver the sample and/or the reagents to the matrix, in order to enable the volume of sample and/or reagents in the receptacle to be measured, and to enable the volume of sample and/or reagents delivered to the matrix to be controlled. In some preferred embodiments, the camera system comprises a refraction target, which provides increased contrast between the background of the image and the meniscus of the liquid (analyte) in the receptacle. The refraction target facilitates the measurements of the volumes directly in the receptacle. In some embodiments, the refraction target is a vertical, dark colour slip placed horizontally behind the receptacle, in the camera-receptacle-refraction target axis. An exemplary set up of the camera system is provided in FIG. 3A. In other preferred embodiments, the camera system does not comprise a refraction target. In such other preferred embodiments, a basic level (or line) detection is used to determine the position of the meniscus of the liquid in the sample receptacle and thus determine the volume of the liquid in the sample receptacle. In some embodiments the means for measuring the volume of analyte in any receptacle of the plurality of receptacles further comprises means for measuring the flow rate of the reagents and/or the sample through the device such that the volume of the analyte in the receptacle can be determined from the flow rate measurements.

In some embodiments, the extraction unit comprises a nozzle configured to force the sample and/or the reagents through the matrix. To force the sample and/or the reagent through the matrix a positive pressure is provided on the analyte in the receptacle. In some embodiments, the positive pressure provided on the analyte is from +1 kPa to +100 kPa. In some embodiments, the nozzle of the extraction unit can form an airtight seal with any receptacle of the plurality of receptacles. The airtight seal enables for the pressure in the receptacle to be increased such that the analyte can be forced through the matrix. In some embodiments, the extraction unit comprises a linear actuator (e.g. an electric or pneumatic actuator) for docking the nozzle to the receptacle.

In some embodiments, the device comprises a valve for releasing and/or maintaining positive pressure, optionally a solenoid valve. The valve can be used to control the flow rate of the analyte through the matrix, by decreasing the air pressure in the receptacle and thus stopping the analyte from moving through the matrix.

In some embodiments, the nozzle is further configured to measure the air pressure in the receptacle. In some embodiments, the air pressure measurements provided by the nozzle are used together with the volume measurements to control the activity of the pumps. In some embodiments, the air pressure measurements are used to determine the presence of a leftover, undesired sample and/or reagent on the matrix. In some embodiments, the presence of the leftover, undesired sample and/or reagent on the matrix is determined using a backpressure measurement. In some embodiments, the backpressure measurement is used to adjust the number of wash cycles required to remove the leftover, undesired sample and/or reagent from the matrix. In some embodiments, the backpressure measurements are used to adjust a time for which the positive pressure is provided to force the sample and/or the reagents through the matrix, without the leftover, undesired sample and/or reagent staying on the matrix.

In some embodiments, the device comprises an in-line sample lysis module for lysis of any sample of the plurality of samples. In some embodiments, the samples are lysed prior to delivery to the device. In some embodiments, the samples are lysed after delivery to the device. Methods for lysis of samples for nucleic acid extraction are well known in the art. In some embodiments, the in-line sample lysis module comprises a mechanical vibration device for mixing of the sample with a lysis buffer. In some embodiments, the in-line sample lysis module comprises a load cell for measuring the volumes of the sample and the lysis buffer that are mixed together. In some embodiments, the lysis can be performed directly in the receptacle, by mixing of the sample and the lysis buffer in the receptacle. In some embodiments, the in-line sample lysis module comprises a magnetic stirrer.

In some embodiments, the device comprises a waste reservoir for collection of waste liquid.

In some embodiments, the nucleic acid is DNA or RNA. In some embodiments, the nucleic acid is DNA. In some embodiments, the nucleic acid is RNA. The skilled person will recognize that depending on the type of nucleic acid to be extracted by the device the reagents for the extraction can be selected from the plurality of reagents well-known in the art. Examples of the reagents for nucleic acid extractions are described in for example in Sambrook et al., Molecular Cloning: A Laboratory Manual (4 ed.), Cold Spring Harbor Laboratory Press, Cold Spring Harbor, N.Y., USA (2012).

A number of matrices for nucleic acid binding is known in the art. In some embodiments, the matrix is a silica matrix (optionally a silica membrane), a glass fibre matrix or a zeolite. In preferred embodiments the matrix is a silica membrane.

In some embodiments, the plurality of receptacles is a plurality of columns. In some embodiments, the plurality of receptacles is a plurality of spin columns. In preferred embodiments, the plurality of receptacles is a plurality of minispin columns. Minispin columns comprising silica membranes for nucleic acid extractions are well known in the art, available commercially and cost-effective.

The number of receptacles in the plurality of receptacles can vary depending on the desired sample-processing capacity of the device. In some embodiments, the plurality of receptacles is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 receptacles.

In some embodiments, the means for housing the plurality of receptacles is a cassette. In preferred embodiments of the invention, the means for housing the plurality of receptacles is a carousel. In some embodiments, the means for housing the plurality of receptacles can be a conveyor belt. In a preferred embodiment, tubing used to deliver the nucleic acid sample is manufactured form an inert material, which nucleic acid molecules will not stick to. In some preferred embodiments, the carousel is configured to move in a single plane (through rotation). In other embodiments, the carousel is configured to move in two planes, the planes being perpendicular to each other.

The skilled person will recognize that a number of pump types can be comprised by the at least one pump. In some embodiments, the at least one pump comprises at least one peristaltic pump. In preferred embodiments the at least one pump comprises at least one syringe pump. In particularly preferred embodiments, the at least one pump comprises at least one diaphragm pump. In some embodiments, the at least one pump comprises at least one metering pump. In some embodiments, the at least one pump is at least one pump in communication with a valve terminal. In some embodiments, the at least one pump comprises at least one pump in communication with a valve terminal. In some embodiments, the valve terminal comprises a plurality of solenoids.

The positive air pressure provided on the analyte in the receptacle may be generated by any suitable means for generating positive air pressure. In some embodiments, the positive air pressure is generated by the at least one pump. In some embodiments, the positive air pressure provided on the analyte in the receptacle is generated by the at least one syringe pump. In preferred embodiments, the positive air pressure provided on the analyte in the receptacle is generated by the at least diaphragm pump. In other embodiments, the positive air pressure provided on the analyte is provided by means for storing compressed air, for example by a compressed air tank.

In some embodiments, the device of the first aspect of the invention is further configured to regenerate any matrix of the plurality of matrices to enable more than one extraction from any matrix of the plurality of matrices. The regeneration of any matrix of the plurality of matrices further increases the sample-processing capacity of the device.

In some embodiments, the device of the first aspect of is further characterized in that:
i) the sample module further comprises a regeneration unit configured to regenerate the matrix in any receptacle of the plurality of receptacles,
ii) the multi-channel module further comprises a plurality of reservoirs for storing reagents for regeneration of the matrix,
iii) the at least one pump is further configured to deliver the reagents for regeneration of the matrix to and/or through the matrix of any receptacle of the plurality of samples, and:
the multi-channel pump module and the sample module are further connected to enable regeneration of any matrix of the plurality of receptacles, and
the means for housing the plurality of receptacles is further configured such that any of the plurality of the receptacles can be positioned for delivery of the reagents from the regeneration unit.

In some embodiments, the extraction unit and the regeneration unit are part of the same unit. In other embodiments, the extraction unit and the regeneration unit are separate. In some embodiments, the regeneration unit comprises a plurality of tubes configured to deliver the reagents to the matrix.

In some embodiments, the regeneration unit comprises a nozzle which is identical or similar to the nozzle of the extraction unit. In some embodiments, the regeneration unit comprises a nozzle configured to force the sample and/or the reagents through the matrix. To force the sample and/or the reagent through the matrix a positive pressure is provided on the analyte in the receptacle. In some embodiments, the positive pressure provided on the analyte is from +1 kPa to +100 kPa. In some embodiments, the device comprises a valve for releasing and/or maintaining positive pressure, optionally a solenoid valve. The valve can be used to control the flow rate of the analyte through the matrix, by decreasing the air pressure in the receptacle and thus stopping the analyte from moving through the matrix. In some embodiments, the nozzle of the regeneration unit can form an airtight seal with any receptacle of the plurality of receptacles. The airtight seal enables for the pressure in the receptacle to be increased such that the analyte can be forced through the matrix. In some embodiments, the nozzle is further configured to measure the air pressure in the receptacle. In some embodiments, the air pressure measurements provided by the nozzle are used together with the volume measurements to control the activity of the pumps. In some embodiments, the air pressure measurements are used to determine the presence of a leftover, undesired sample and/or reagent on the matrix. In some embodiments, the presence of the leftover, undesired sample and/or reagent on the matrix is determined using a backpressure measurement. In some embodiments, the backpressure measurement is used to adjust the number of wash cycles required to remove the leftover, undesired sample and/or reagent from the matrix. In some embodiments, the backpressure measurements are used to adjust a time for which the positive pressure is provided to force the sample and/or the reagents through the matrix, without the leftover, undesired sample and/or reagent staying on the matrix. In some embodiments, the regeneration unit comprises a linear actuator (e.g. an electric or pneumatic actuator) for docking the nozzle to the receptacle.

The reagents for regeneration of the matrix for nucleic acid binding are known in the art. The skilled person will recognize that the reagents for regeneration of the matrix for nucleic acid binding can comprise any reagents for chemical or non-chemical regeneration of the matrix for nucleic acid binding known in the art. In some embodiments, the reagents for chemical regeneration of the matrix comprise a hypochlorite. In some embodiments of the invention, the reagents for chemical regeneration of the matrix comprise sodium hypochlorite, preferably 0.7% sodium hypochlorite. In some embodiments, the regeneration reagent is any suitable cleaning reagent such as bleach, water, PBS, borate buffers, ethanol, isopropyl alcohol (IPA) and DNAZap™ PCR DNA. In some embodiments the regeneration reagent is selected from bleach, water, PBS, borate buffers, ethanol, isopropyl alcohol (IPA) and DNAZap™ PCR DNA Degradation reagent.

In other embodiments, a cleaning solution for regenerating the matrix for nucleic acid binding does not comprise bleach-based reagents. In some embodiments, a cleaning solution comprises water, PBS and/or borate buffers. In such embodiments, the regeneration of the matrix is achieved by washing the membrane resulting in removal of the nucleic acid bound to the matrix.

In some embodiments, the plurality of tubes of the device comprises projections (e.g. pipette tips), narrowing towards the end of the projections such that fine droplets can be created when a liquid (e.g. the samples or reagents) is delivered through the plurality of tubes.

In some embodiments, the device of the invention comprises at least one actuator. In some embodiments, the device comprises 1, 2, 3, 4, 5, 6, 7, 8, 9 or 10 actuators. In some embodiments at least one actuator is at least one electric actuator. In other embodiments, the at least one actuator is at least one pneumatic actuator. In further embodiments the device comprises at least one linear actuator and at least one pneumatic actuator.

In some embodiments the device comprises an actuator configured to lower the end of the tube for delivery of any of the reagents and/or sample into the receptacle during delivery. Such lowering allows to prevent excessive splashing during delivery into the receptacle.

In some embodiments, the device comprises a suction system. The suction system comprises a suction pump, a waste reservoir for collection of waste liquid and a linear actuator configured to dock the waste reservoir of the suction system to the receptacle. Optionally, the waste reservoir is docked to the receptacle using a funnel. The docking of the waste reservoir of the suction system to the sample receptacle is not air-tight. The use of the suction system during operation of the device prevents excessive aerosols from contaminating the device, when sample and/or reagents are being provided through the matrix of the receptacle using positive pressure.

In the second aspect of the invention, a process for automated, matrix-based extraction of nucleic acid samples from a plurality of samples comprises the steps of:
a) receiving a sample of the plurality of samples in a receptacle of a plurality of receptacles,
b) binding a nucleic acid to a matrix,
c) washing the matrix to remove residual reagent, and
d) eluting the nucleic acid from the matrix;
   wherein a volume of sample used in the process and/or a volume of any reagent of a plurality of reagents used in the process are controlled using image processing; and
   wherein image processing is used to measure the volume of the sample and/or the volume of any reagent of a plurality of reagents received in the receptacle.

Advantageously, the use of the image processing for volume measurements directly in the receptacle enables for the process to be controlled and adjusted such that the accuracy and precision of the volumes used in the process of the invention does not change with time and exploitation of the devices used in the process. The processes for automated, matrix-based extraction of nucleic acids known in the art predominantly relying on the use of either pipetting or syringe pumps for measuring the volume of the reagents suffer from the need for maintenance of the device to ensure the accuracy and precision of the measured volumes. Measuring the volume of the sample and/or the reagent received in the receptacle enables for feedback to be used in the process such that the volume of the sample and/or the reagent can be readily adjusted to prevent decrease in the accuracy and precision of the volume of the reagents used in the process. Additionally, the use of volume measurements directly in the receptacle overcomes the issue wherein the air inside the syringe pump is expanded and compressed during the pumping process, resulting in lowered accuracy and precision of the volume delivered. The extent of air compression and expansion in the syringe pumps is also likely to change over time, resulting in further decreases in accuracy and precision of the volume delivered, which is again overcome with the use of volume measurements directly in the receptacle. In some embodiments, the control of the volume of the reagents is performed continuously. In preferred embodiments, the control of the volume of the reagents is performed using closed loop feedback control. In some embodiments, in addition to the use of the image processing for volume measurements, the process of the invention comprises using flow rate measurements of the reagent and/or samples throughout the process, in order to determine the volume of the sample and/or the volume of any reagent received in the receptacle.

In some embodiments, the process comprises using a positive pressure to force the sample and/or the reagent through the matrix. The skilled person will recognize that a range of pressure can be used in the process to force the sample and/or the reagent through the matrix. In some embodiments, the positive pressure is from +1 kPa to +100 kPa, in some embodiments from +20 kPa to +50 kPa, e. g +20 kPa, +30 kPa, +40 kPa or +50 kPa. The positive air pressure provided on the analyte in the receptacle may be generated by any suitable means for generating positive air pressure. In some embodiments, the positive air pressure is generated by at least one syringe pump. In preferred embodiments, the positive air pressure is generated by the at least diaphragm pump. In other embodiments, the positive air pressure provided on the analyte is provided by means for storing compressed air, for example by a compressed air tank.

In some embodiments, a presence of the leftover, undesired sample and/or reagent on the matrix is determined using an air pressure measurement in the receptacle. In preferred embodiments the presence of the leftover, undesired sample and/or reagent on the matrix is determined using a backpressure measurement. In some further preferred embodiments, the backpressure measurement is used to adjust the number of wash cycles required to remove the leftover, undesired sample and/or reagent from the matrix. In some embodiments, the backpressure measurements are used to adjust a time for which the positive pressure is provided to force the sample and/or the reagents through the matrix, without the leftover, undesired sample and/or reagent staying on the matrix.

A number of matrices for nucleic acid binding which can be used in the process is known in the art. In some embodiments, the matrix is a silica matrix (optionally a silica membrane), a glass fibre matrix or a zeolite. In preferred embodiments the matrix is a silica membrane. In some embodiments, the silica membrane is comprised by a column. In some embodiments, the silica membrane is comprised by a spin column. In preferred embodiments, the silica membrane is comprised by a minispin column.

In the third aspect of the invention, the device for automated extraction of nucleic acid samples from a plurality of samples, comprises:
i) a sample module comprising:
   a) a plurality of receptacles for receiving the plurality of samples, wherein each receptacle of the plurality of receptacles comprises a matrix for nucleic acid binding,
   b) means for housing the plurality of receptacles,
   c) an extraction unit configured to extract the nucleic acid samples from any sample of the plurality of samples, and
   d) a regeneration unit configured to regenerate the matrix in any receptacle of the plurality of receptacles; and
ii) a multi-channel pump module, comprising:
   e) a plurality of reservoirs for storing reagents for extraction of nucleic acid samples and a plurality of reservoirs for storing reagents for regeneration of the matrix, and
   f) at least one pump configured to deliver the plurality of samples, the reagents for extraction of nucleic acid samples and/or the reagents for regeneration of the matrix to and/or through the matrix of any receptacle of the plurality of receptacles; and wherein:
      the multi-channel pump module and the sample module are connected such that the reagents can be delivered to and/or through any matrix of the plurality of receptacles to enable the extraction of the nucleic acid samples from the plurality of samples and such that any matrix of the plurality of receptacles can be regenerated, and
      the means for housing the plurality of receptacles is configured such that any of the plurality of the receptacles can be positioned for delivery of the reagents from the extraction unit or the regeneration unit.

In some embodiments, the at least one pump is a single pump. In some embodiments, the at least one pump is a plurality of pumps.

In some embodiments, the extraction unit and the regeneration unit are part of the same unit. In other embodiments, the extraction unit and the regeneration unit are separate. The regeneration unit increases the sample-processing capacity of the device without the need for increasing the number of the receptacles and/or matrices comprised by the device. The devices known in the art are characterized by sample processing capacity being dependent on the number of receptacles and/or matrices used, with one disposable matrix being used for extraction of nucleic acids from a single sample only in majority of said known devices. The devices which do use matrix regeneration, described for example in Preston C. et al. 2011 “Underwater application of quantitative PCR on an ocean mooring”, not only use a single receptacle, but also require the use of custom-designed receptacles with matrices for nucleic acid binding, which increases the cost involved with production of the device. The use of a single matrix, even with matrix-regeneration capacity provided, results in the device being unable to operate in situations where the single matrix stops working because of e.g., a mechanical failure (such as for example being clogged by non-filtered particulate in the sample provided to the matrix). Increasing the sample processing capacity by providing an increasingly larger number of disposable matrices in the device is undesirable and limited for several reasons, e.g., frequent necessity for waste disposal, high operating cost and increasing the footprint required for the process to operate with the desired processing capacity. By providing a matrix regeneration capability in the device, the sample processing capacity of the device is increased, which allows for the device to operate for longer without the need for providing replacement matrices and/or receptacles to the device. It further limits the need for providing waste disposal in the device as the matrices can be used for multiple extraction cycles prior to being eventually disposed of.

The reagents for regeneration of the matrix for nucleic acid binding are known in the art. The skilled person will recognize that the reagents for regeneration of the matrix for nucleic acid binding can comprise any reagents for chemical regeneration of the matrix for nucleic acid binding known in the art. In some embodiments, the reagents for chemical regeneration of the matrix comprise a hypochlorite. In preferred embodiments of the invention, the reagents for chemical regeneration of the matrix comprise sodium hypochlorite, preferably 0.7% sodium hypochlorite. In some embodiments, the regeneration reagent is any suitable cleaning reagent such as bleach, water, PBS, borate buffers, ethanol, isopropyl alcohol (IPA) and DNAZap™ PCR DNA. In some embodiments the regeneration reagent is selected from bleach, water, PBS, borate buffers, ethanol, isopropyl alcohol (IPA) and DNAZap™ PCR DNA Degradation reagent.

In some embodiments, the reagents for regeneration of the matrix for nucleic acid binding comprise water, PBS or borate buffers. In such embodiments, the regeneration of the matrix is achieved by washing the membrane resulting in removal of the nucleic acid bound to the matrix.

In some embodiments, the extraction unit and the regeneration unit each comprise a plurality of tubes configured to deliver the sample and/or the reagents to the matrix. In some embodiments, the extraction unit and the regeneration unit each comprise a nozzle configured to force the sample and/or the reagents through the matrix. To force the sample and/or the reagent through the matrix a positive pressure is provided on the analyte in the receptacle. In some embodiments, the positive pressure provided on the analyte is from +1 kPa to +100 kPa, in some embodiments from +20 kPa to +50 kPa, e.g. +20 kPa, +30 kPa, +40 kPa or +50 kPa. In some embodiments, the device comprises a valve for releasing and/or maintaining positive pressure, optionally a solenoid valve. The valve can be used to control the flow rate of the analyte through the matrix, by decreasing the air pressure in the receptacle and thus stopping the analyte from moving through the matrix. In some embodiments, the nozzle of the extraction unit and the nozzle of the regeneration unit can each form an airtight seal with any receptacle of the plurality of receptacles. The airtight seal enables for the pressure in the receptacle to be increased such that the analyte can be forced through the matrix. In some embodiments, the extraction unit and/or the regeneration unit each comprise at least one linear actuator (e.g. an electric or pneumatic actuator) for docking each nozzle to the receptacle.

In some embodiments, the device comprises an in-line sample lysis module for lysis of any sample of the plurality of samples. In some embodiments, the samples are lysed prior to delivery to the device. In some embodiments, the samples are lysed after delivery to the device. Methods for lysis of samples for nucleic acid extraction are well known in the art. In some embodiments, the in-line sample lysis module comprises a mechanical vibration device for mixing of the sample with a lysis buffer. In some embodiments, the in-line sample lysis module comprises a load cell for measuring the volumes of the sample and the lysis buffer that are mixed together. In some embodiments, the lysis can be performed directly in the receptacle, by mixing of the sample and the lysis buffer in the receptacle. In some embodiments, the in-line sample lysis module comprises a magnetic stirrer.

In some embodiments, the device comprises a waste reservoir for collection of waste liquid.

In some embodiments, the nucleic acid is DNA or RNA. In some embodiments, the nucleic acid is DNA. In some embodiments, the nucleic acid is RNA. The skilled person will recognize that depending on the type of nucleic acid to be extracted by the device the reagents for the extraction can selected from the plurality of reagents well-known in the art. Examples of the reagents for nucleic acid extractions are described in for example in Sambrook et al., Molecular Cloning: A Laboratory Manual (4 ed.), Cold Spring Harbor Laboratory Press, Cold Spring Harbor, N.Y., USA (2012).

A number of matrices for nucleic acid binding is known in the art. In some embodiments, the matrix is a silica matrix (optionally a silica membrane), a glass fibre matrix or a zeolite. In preferred embodiments the matrix is a silica membrane.

In some embodiments, the plurality of receptacles is a plurality of columns. In some embodiments, the plurality of receptacles is a plurality of spin columns. In preferred embodiments, the plurality of receptacles is a plurality of minispin columns. Minispin columns comprising silica membranes for nucleic acid extractions are well known in the art, available commercially and cost-effective.

The number of receptacles in the plurality of receptacles can vary depending on the desired sample-processing capacity of the device. In some embodiments, the plurality of receptacles is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 receptacles

In some embodiments, the means for housing the plurality of receptacles is a cassette. In preferred embodiments of the invention, the means for housing the plurality of receptacles is a carousel. In a preferred embodiment, tubing used to deliver the nucleic acid sample is manufactured form an inert material, which nucleic acid molecules will not stick to. In some preferred embodiments, the carousel is configured to move in a single plane (through rotation). In other embodiments, the carousel is configured to move in two planes, the planes being perpendicular to each other.

The skilled person will recognize that a number of pump types can be comprised by the at least one pump. In some embodiments, the at least one pump comprises at least one peristaltic pump. In preferred embodiments the at least one pump comprises at least one syringe pump. In particularly preferred embodiments, the at least one pump comprises at least one diaphragm pump. In some embodiments, the at least one pump comprises at least one metering pump. In some embodiments, the at least one pump is at least one pump in communication with a valve terminal. In some embodiments, the at least one pump comprises at least one pump in communication with a valve terminal. In some embodiments, the valve terminal comprises a plurality of solenoids.

The positive air pressure provided on the analyte in the receptacle may be generated by any suitable means for generating positive air pressure. In some embodiments, the positive pressure is generated by the at least one pump. In some embodiments, the positive air pressure provided on the analyte in the receptacle is generated by the at least one syringe pump. In preferred embodiments, the positive air pressure provided on the analyte in the receptacle is generated by the at least one diaphragm pump. In other embodiments, the positive air pressure provided on the analyte is provided by means for storing compressed air, for example by a compressed air tank.

In some embodiments, the device comprises means for measuring a volume of the analyte in any receptacle of the plurality of receptacles. In some embodiments, the volume measurement provided by the means for measuring the volume of the analyte is used to control the at least one pump. In particularly preferred embodiments, the volume measurement is used to control the at least one pump using a closed loop feedback system. In some preferred embodiments, the control of the at least one pump is performed periodically. In particularly preferred embodiments, the control of the at least one pump is performed continuously. In some embodiments, the control of the at least one pump using the volume measurement allows for the adjustment of pump activity to deliver the sample and/or reagent through the matrix.

The skilled person will recognize that a plurality of embodiments can be employed to provide means for measuring the volume of analyte in any receptacle of the plurality of receptacles. In some embodiments, the means for measuring the volume of analyte in any receptacle of the plurality of receptacles comprises a weight-measurement system. In some embodiments, the means for measuring the volume of analyte in any receptacle of the plurality of receptacles comprises a load cell. In some embodiments, the means for measuring the volume of analyte in any receptacle of the plurality of receptacles comprises a sound-based system, optionally an ultrasound-based system. In other embodiments, the means for measuring the volume of analyte in any receptacle of the plurality of receptacles comprises a camera system. In some embodiments of the invention the camera system can be positioned in sufficiently close proximity to the plurality of tubes configured to deliver the sample and/or the reagents to the matrix, in order to enable the volume of sample and/or reagents in the receptacle to be measured, and to enable the volume of sample and/or reagents delivered to the matrix to be controlled. In some embodiments, the camera system comprises a refraction target, which provides increased contrast between the background of the image and the meniscus of the liquid (analyte) in the receptacle. The refraction target facilitates the measurements of the volumes directly in the receptacle. In some embodiments, the refraction target is a vertical, dark colour slip placed horizontally behind the receptacle, in the camera-receptacle-refraction target axis. An exemplary set up of the camera system is provided in FIG. 3A. In other embodiments, the camera system does not comprise a refraction target. In such other embodiments, a basic level (or line) detection is used to determine the position of the meniscus of the liquid in the sample receptacle and thus determine the volume of the liquid in the sample receptacle. In some embodiments the means for measuring the volume of analyte in any receptacle of the plurality of receptacles further comprises means for measuring the flow rate of the reagents and/or the sample through the device such that the volume of the analyte in the receptacle can be determined from the flow rate measurements.

In some embodiments, the nozzle of the extraction unit and the nozzle of the regeneration unit are further configured to measure the air pressure in the receptacle. In some embodiments, the air pressure measurements provided by the nozzle of the extraction unit and/or the nozzle of the regeneration unit are used together with the volume measurements to control the activity of the pumps. In some embodiments, the air pressure measurements are used to determine the presence of a leftover, undesired sample and/or reagent on the matrix. In some embodiments, the presence of the leftover, undesired sample and/or reagent on the matrix is determined using a backpressure measurement. In some embodiments, the backpressure measurement is used to adjust the number of wash cycles required to remove the leftover, undesired sample and/or reagent from the matrix.

In some embodiments, the plurality of tubes of the device comprises projections (e.g. pipette tips), narrowing towards the end of the projections such that fine droplets can be created when a liquid (e.g. the samples or reagents) is delivered through the plurality of tubes.

In some embodiments, the device of the invention comprises at least one actuator. In some embodiments, the device comprises 1, 2, 3, 4, 5, 6, 7, 8, 9 or 10 actuators. In some embodiments at least one actuator is at least one electric actuator. In other embodiments, the at least one actuator is at least one pneumatic actuator. In further embodiments the device comprises at least one linear actuator and at least one pneumatic actuator.

In some embodiments the device comprises an actuator configured to lower the end of the tube for delivery of any of the reagents and/or sample into the receptacle during delivery. Such lowering allows to prevent excessive splashing during delivery into the receptacle.

In some embodiments, the device comprises a suction system. The suction system comprises a suction pump, a waste reservoir for collection of waste liquid and a linear actuator configured to dock the waste reservoir of the suction system to the receptacle. Optionally, the waste reservoir is docked to the receptacle using a funnel. The docking of the waste reservoir of the suction system to the sample receptacle is not air-tight. The use of the suction system during operation of the device prevents excessive aerosols from contaminating the device, when sample and/or reagents are being provided through the matrix of the receptacle using positive pressure.

In the fourth aspect of the invention, the process for automated, matrix-based extraction of nucleic acid samples from a plurality of samples using a plurality of matrices for nucleic acid binding comprises a nucleic acid extraction step and a matrix regeneration step, wherein the nucleic acid extraction step and the matrix regeneration step are performed in-parallel.

The processes known in the art are characterized by sample processing capacity being dependent on the number of receptacles and/or matrices used, with one disposable matrix being used for extraction of nucleic acids from a single sample only in majority of said processes. The processes which do use matrix regeneration, described for example in Preston C. et al. 2011 “Underwater application of quantitative PCR on an ocean mooring”, not only use a single column, but also require the use of custom-designed receptacles with matrices for nucleic acid binding, which increases the cost involved with the process. Increasing the sample processing capacity by providing an increasingly larger number of disposable matrices in the process is undesirable for several reasons, e.g., frequent necessity for waste disposal, high operating cost and increasing the footprint required for the process to operate with the desired processing capacity. By using a matrix regeneration step in parallel to the nucleic acid extraction step in the processes, the sample processing capacity of the process is increased, which allows for the process to be performed for longer without the need for providing replacement matrices and/or receptacles during the process. It further limits the need for providing waste disposal in the process as the matrices can be used for multiple extraction cycles prior to being eventually disposed of.

The reagents for regeneration of the matrix for nucleic acid binding are known in the art. The skilled person will recognize that the reagents for regeneration of the matrix for nucleic acid binding can comprise any reagents for chemical regeneration of the matrix for nucleic acid binding known in the art. In some embodiments, the reagents for chemical regeneration of the matrix comprise a hypochlorite. In preferred embodiments of the invention, the reagents for chemical regeneration of the matrix comprise sodium hypochlorite, preferably 0.7% sodium hypochlorite. In some embodiments, the regeneration reagent is any suitable cleaning reagent such as bleach, water, PBS, borate buffers, ethanol, isopropyl alcohol (IPA) and DNAZap™ PCR DNA. In some embodiments the regeneration reagent is selected from bleach, water, PBS, borate buffers, ethanol, isopropyl alcohol (IPA) and DNAZap™ PCR DNA Degradation reagent.

In other embodiments, a cleaning solution for regenerating the matrix for nucleic acid binding does not comprise bleach-based reagents. In some embodiments, the reagents for regeneration of the matrix for nucleic acid binding comprise water, PBS or borate buffers. In such embodiments, the regeneration of the matrix is achieved by washing the membrane resulting in removal of the nucleic acid bound to the matrix.

In some embodiments, the regeneration step comprises contacting the matrix with a regeneration reagent, followed by washing the matrix with water. In some exemplary embodiments, the volume of 0.7% sodium hypochlorite used for regeneration is 0.5 mL. In some exemplary embodiments, the volume of water used for the washing is 20 mL.

In some embodiments, a presence of the leftover, undesired sample and/or reagent on the matrix is determined using an air pressure measurement in the receptacle. In preferred embodiments the presence of the leftover, undesired sample and/or reagent on the matrix is determined using a backpressure measurement. In some further preferred embodiments, the backpressure measurement is used to adjust the number of wash cycles required to remove the leftover, undesired sample and/or reagent from the matrix.

A number of matrices for nucleic acid binding which can be used in the process is known in the art. In some embodiments, the matrix is a silica matrix (optionally a silica membrane), a glass fibre matrix or a zeolite. In preferred embodiments the matrix is a silica membrane. In some embodiments, the silica membrane is comprised by a column. In some embodiments, the silica membrane is comprised by a spin column. In preferred embodiments, the silica membrane is comprised by a minispin column.

It will be recognized that the sample processing capacity of the process will be a function of the number of matrices in the plurality of matrices used in the process and the number of times each matrix of the plurality of matrices is regenerated in the process.

In some embodiments, the plurality of matrices is selected from at least 2 matrices, at least 3 matrices, at least 4 matrices, at least 5 matrices, at least 6 matrices, at least 7 matrices, at least 8 matrices, at least 9 matrices, at least 10 matrices, at least 11 matrices, at least 12 matrices, at least 13 matrices, at least 14 matrices, at least 15 matrices, at least 16 matrices, at least 17 matrices, at least 18 matrices, at least 19 matrices, at least 20 matrices, at least 30 matrices, at least 40 matrices, at least 50 matrices, at least 60 matrices, at least 70 matrices, at least 80 matrices, at least 90 matrices or at least 100 matrices.

In some embodiments, each matrix of the plurality of matrices is regenerated at least 1 time. In some embodiments, each matrix of the plurality of matrices is regenerated at least 2 times. In some embodiments, each matrix of the plurality of matrices is regenerated at least 3 times. In some embodiments, each matrix of the plurality of matrices is regenerated at least 4 times. In some embodiments, each matrix of the plurality of matrices is regenerated at least 5 times. In some embodiments, each matrix of the plurality of matrices is regenerated at least 6 times. In some embodiments, each matrix of the plurality of matrices is regenerated at least 7 times. In some embodiments, each matrix of the plurality of matrices is regenerated at least 8 times. In some embodiments, each matrix of the plurality of matrices is regenerated at least 9 times. In some embodiments, each matrix of the plurality of matrices is regenerated at least 10 times.

### EXEMPLARY EMBODIMENTS

### Exemplary Embodiment 1

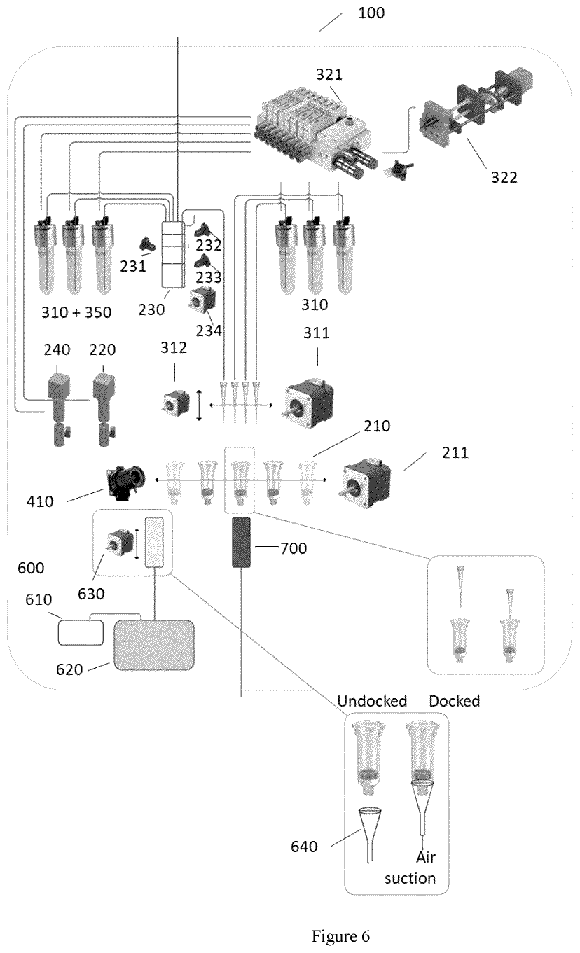

An exemplary embodiment of the device of the invention is provided in FIG. 6. The exemplary embodiment of the device for automated extraction of nucleic acid samples from a plurality of samples, comprises:
i) a sample module (200) comprising:
   a) a plurality of receptacles (210) for receiving the plurality of samples, wherein each receptacle of the plurality of receptacles comprises a matrix for nucleic acid binding,
   b) means for housing the plurality of receptacles, and
   c) an extraction unit (220) configured to extract the nucleic acid samples from any sample of the plurality of samples; and
ii) a multi-channel pump module (300), comprising:
   d) a plurality of reservoirs (310) for storing reagents for extraction of nucleic acid samples, and
   e) at least one pump (320) configured to deliver the plurality of samples and/or the reagents for extraction of nucleic acid samples to and/or through the matrix of any receptacle of the plurality of receptacles; and wherein:
      the device comprises means (400) for measuring the volume of analyte in any receptacle of the plurality of receptacles (for example a camera system (410),
      the multi-channel pump module and the sample module are connected such that the reagents can be delivered to and/or through any matrix of the plurality of receptacles to enable the extraction of the nucleic acid samples from the plurality of samples, and
      the means for housing the plurality of receptacles is configured such that any of the plurality of the receptacles can be positioned for delivery of the reagents from the extraction unit.

In the exemplary embodiment, the sample module further comprises a regeneration unit (240) configured to regenerate the matrix in any receptacle of the plurality of receptacles,
ii) the multi-channel module further comprises a plurality of reservoirs (350) for storing reagents for regeneration of the matrix,
iii) the at least one pump is further configured to deliver the reagents for regeneration of the matrix to and/or through the matrix of any receptacle of the plurality of samples, and
the multi-channel pump module and the sample module are further connected to enable regeneration of any matrix of the plurality of receptacles, and
the means for housing the plurality of receptacles is further configured such that any of the plurality of the receptacles can be positioned for delivery of the reagents from the regeneration unit.

In the exemplary embodiment, the sample module further comprises an in-line sample lysis module (230) for mixing of the sample with a lysis buffer. Filling level sensors (231-233) determine the filling levels of the in-line sample lysis module. An actuator (234) is configured to drive a magnetic stirrer comprised by the in-line sample lysis module. An actuator (211) is configured to select any receptacle of the plurality of receptacles for delivery.

In the exemplary embodiment, the multi-channel pump module further comprises a (solenoid) valve terminal (321) and the at least one pump is a syringe pump (322) in communication with the valve terminal. The valve terminal comprises a plurality of solenoids. The at least one pump and the valve terminal are in communication with the plurality of reservoirs. The at least one pump provides positive pressure to the plurality of reagent reservoirs, such that the reagents can be delivered to the plurality of receptacles. An actuator (311) is configured to select any reagent of the samples and/or reagents for delivery. An actuator (312) is configured to move a plurality of tubes of the device vertically, in order to prevent excessive splashing during delivery of the reagent and/or the sample. In the example, the plurality of tubes comprises projections (e.g. pipette tips), narrowing towards the end of the projections such that fine droplets can be created when a liquid (e.g. the samples or reagents) is delivered through the plurality of tubes. The projections can be integrated into the end of the tube or detachable.

The at least one pump and the valve terminal are also in communication with the extraction unit such that positive pressure can be provided to the extraction unit in order for the sample and/or the reagents to be delivered through the matrix. The at least one pump and the valve terminal are also in communication with the in-line sample lysis module such that the sample can be delivered from the in-line sample lysis module to the sample receptacle. The at least one pump and the valve terminal are also in communication with the plurality of reservoirs for storing reagents for regeneration of the matrix and the in-line sample lysis module such that the in-line sample lysis module can be cleaned using the reagents for regeneration of the matrix. The at least one pump and the valve terminal are also in communication with the regeneration unit such that positive pressure can be provided to the regeneration unit in order for the regeneration reagents to be delivered through the matrix.

The use of positive pressure provided by the at least one pump and the valve terminal for delivering positive pressure through the extraction and/or regeneration unit and through the matrices of the sample receptacles circumvents the need for conventional use of vacuum or centrifugal force to move the reagents and/or samples through the matrices. However, pressurizing the matrix (e.g. a silica membrane) leads to generation of aerosols when the liquids (i.e. the reagents and/or the sample) leave the matrix. In particular, lysis reagents used for nucleic acid extraction can be harmful to the device handlers and/or result in damage of the device components over time. To limit the generation of aerosols, in some embodiments the device further comprises a suction system (600) comprising a suction pump (610), a waste reservoir (620) for collection of waste liquid and a linear actuator (630). The linear actuator is configured to dock the waste reservoir of the suction system to the sample receptacle (for example using a funnel (640)), such that aerosols are sucked into the waste reservoir. The docking of the waste reservoir to the sample receptacle is not air-tight, in order to prevent the negative pressure created by the suction pump from causing the movement of the reagent and/or sample through the matrix of the receptacle.

In the exemplary embodiment, the nucleic acid following extraction is collected in a nucleic acid collection container (700). Preferably the nucleic acid container is an RNA collection container.

### Exemplary Embodiment 2

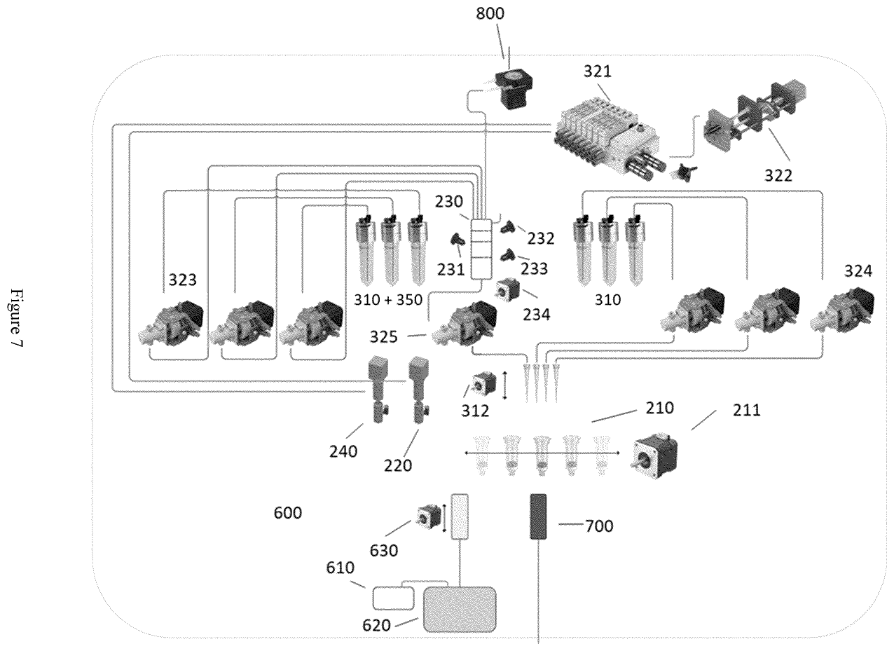

An exemplary embodiment of the device of the invention is provided in FIG. 7. The exemplary embodiment of the device for automated extraction of nucleic acid samples from a plurality of samples, comprises:
i) a sample module (200) comprising:
   a) a plurality of receptacles (210) for receiving the plurality of samples, wherein each receptacle of the plurality of receptacles comprises a matrix for nucleic acid binding,
   b) means for housing the plurality of receptacles, and
   c) an extraction unit (220) configured to extract the nucleic acid samples from any sample of the plurality of samples; and
ii) a multi-channel pump module (300), comprising:
   d) a plurality of reservoirs (310) for storing reagents for extraction of nucleic acid samples, and
   e) at least one pump (320) configured to deliver the plurality of samples and/or the reagents for extraction of nucleic acid samples to and/or through the matrix of any receptacle of the plurality of receptacles; and wherein:
      the device comprises means (400) for measuring the volume of analyte in any receptacle of the plurality of receptacles,
      the multi-channel pump module and the sample module are connected such that the reagents can be delivered to and/or through any matrix of the plurality of receptacles to enable the extraction of the nucleic acid samples from the plurality of samples, and
      the means for housing the plurality of receptacles is configured such that any of the plurality of the receptacles can be positioned for delivery of the reagents from the extraction unit.

In the exemplary embodiment, the sample module further comprises a regeneration unit (240) configured to regenerate the matrix in any receptacle of the plurality of receptacles,
ii) the multi-channel module further comprises a plurality of reservoirs (350) for storing reagents for regeneration of the matrix,
iii) the at least one pump is further configured to deliver the reagents for regeneration of the matrix to and/or through the matrix of any receptacle of the plurality of samples, and
the multi-channel pump module and the sample module are further connected to enable regeneration of any matrix of the plurality of receptacles, and
the means for housing the plurality of receptacles is further configured such that any of the plurality of the receptacles can be positioned for delivery of the reagents from the regeneration unit.

In the exemplary embodiment, the sample module further comprises an in-line sample lysis module (230) for mixing of the sample with a lysis buffer. Filling level sensors (231-233) determine the filling levels of the in-line sample lysis module. An actuator (234) is configured to drive a magnetic stirrer comprised by the in-line sample lysis module. An actuator (211) is configured to select any receptacle of the plurality of receptacles for delivery.

In the exemplary embodiment, the multi-channel pump module further comprises a (solenoid) valve terminal (321) and the at least one pump comprises a syringe pump (322) in communication with the valve terminal. In some embodiments, the valve terminal comprises a plurality of solenoids. The at least one pump further comprises metering pumps (e.g. at least one metering pump (323), at least one metering pump (324) and a metering pump (325)). The at least one metering pump (323) is in communication with the reagent reservoirs (310) and the sample receptacle and is configured to deliver the reagents for extraction of nucleic acids to the receptacle. The at least one metering pump (323) is also in communication with the reagent reservoirs (310) and the in-line sample lysis module. The at least one metering pump (323) is also in communication with the reagent reservoirs (350) and the in-line sample lysis module such that the in-line sample lysis module can be cleaned using the reagents for regeneration of the matrix. The at least one metering pump (324) is in communication with the reservoirs (310) and is configured to deliver the reagents for extraction of nucleic acids to the receptacle. The metering pump (325) is configured to deliver the sample for nucleic acid extraction from the in-line sample lysis module to the sample receptacle. In the exemplary embodiment, the metering pumps provide the means for measuring the volume of analyte. The at least one pump and the valve terminal are in communication with the plurality of reservoirs. An actuator (312) is configured to move a plurality of tubes of the device vertically, in order to prevent excessive splashing during delivery of the reagent and/or the sample. In the example, the plurality of tubes comprises projections (e.g. pipette tips), narrowing towards the end of the projections such that fine droplets can be created when a liquid (e.g. the samples or reagents) is delivered through the plurality of tubes. The projections can be integrated into the end of the tube or detachable.

The single pump and the valve terminal are also in communication with the extraction unit such that positive pressure can be provided to the extraction unit in order for the sample and/or the reagents to be delivered through the matrix. The single pump and the valve terminal are also in communication with the regeneration unit such that positive pressure can be provided to the regeneration unit in order for the regeneration reagents to be delivered through the matrix. The single pump and the valve terminal are also in communication with the in-line sample lysis module such that the in-line sample lysis module can be emptied with pressurized air if needed.

The use of positive pressure provided by the at least one pump and the valve terminal for delivering positive pressure through the extraction and/or regeneration unit and through the matrices of the sample receptacles circumvents the need for conventional use of vacuum or centrifugal force to move the reagents and/or samples through the matrices. However, pressurizing the matrix (e.g. a silica membrane) leads to generation of aerosols when the liquids (i.e. the reagents and/or the sample) leave the matrix. In particular, lysis reagents used for nucleic acid extraction can be harmful to the device handlers and/or result in damage of the device components over time. To limit the generation of aerosols, in some embodiments the device further comprises a suction system (600) comprising a suction pump (610), a waste reservoir (620) for collection of waste liquid and a linear actuator (630). The linear actuator is configured to dock the waste reservoir of the suction system to the sample receptacle (for example using a funnel (640)), such that aerosols are sucked into the waste reservoir. The docking mode is as depicted in FIG. 6. The docking of the waste reservoir to the sample receptacle is not air-tight, in order to prevent the negative pressure created by the suction pump from causing the movement of the reagent and/or sample through the matrix of the receptacle.

In the exemplary embodiment, the nucleic acid following extraction is collected in a nucleic acid collection container (700). Preferably the nucleic acid container is an RNA collection container.

In some embodiments, the device comprises a peristaltic pump (800) to provide the sample from a source to the in-line sample lysis module.

### Numbered Embodiments

1. A device for automated extraction of nucleic acid samples from a plurality of samples, comprising:
   i) a sample module comprising:
      a) a plurality of receptacles for receiving the plurality of samples, wherein each receptacle of the plurality of receptacles comprises a matrix for nucleic acid binding,
      b) means for housing the plurality of receptacles, and
      c) an extraction unit configured to extract the nucleic acid samples from any sample of the plurality of samples; and
   ii) a multi-channel pump module, comprising:
      d) a plurality of reservoirs for storing reagents for extraction of nucleic acid samples, and
      e) a plurality of pumps configured to deliver the plurality of samples and/or the reagents for extraction of nucleic acid samples to and/or through the matrix of any receptacle of the plurality of receptacles; and wherein:
         the device comprises means for measuring the volume of analyte in any receptacle of the plurality of receptacles,
         the multi-channel pump module and the sample module are connected such that the reagents can be delivered to and/or through any matrix of the plurality of receptacles to enable the extraction of the nucleic acid samples from the plurality of samples, and
         the means for housing the plurality of receptacles is configured such that any of the plurality of the receptacles can be positioned for delivery of the reagents from the extraction unit.
2. The device of embodiment 1, wherein the volume measurements are used to control the plurality of pumps, preferably using closed loop feedback control.
3. The device of embodiment 1 or embodiment 2, wherein the means for measuring the volume of analyte is a camera system.
4. The device of any preceding embodiment, wherein the extraction unit comprises a nozzle configured to force the sample and/or the reagents through the matrix.
5. The device of embodiment 4, wherein the nozzle can form an airtight seal with any receptacle of the plurality of receptacles.
6. The device of embodiment 4 or embodiment 5, wherein the nozzle is configured to measure the air pressure in the receptacle.
7. The device of embodiment 6, wherein the pressure measurement is used together with the volume measurements to control the plurality of pumps.
8. The device of any preceding embodiment, wherein the device comprises an in-line lysis module for lysis of any sample of the plurality of samples.
9. The device of embodiment 8, wherein the in-line lysis module comprises a mechanical vibration device for mixing of the sample with a lysis buffer.
10. The device of any preceding embodiment, wherein the nucleic acid is DNA or RNA.
11. The device of any preceding embodiment, wherein the matrix is a silica membrane, a glass fibre matrix or a zeolite.
12. The device of any preceding embodiment, wherein the plurality of receptacles is a plurality of columns, preferably a plurality of mini spin columns.
13. The device of any preceding embodiment, wherein the plurality of receptacles is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 receptacles.
14. The device of any preceding embodiment, wherein the means for housing the plurality of receptacles is a carousel.
15. The device of any preceding embodiment, wherein the plurality of pumps comprises peristaltic pumps.
16. The device of any preceding embodiment, wherein the plurality of pumps comprises syringe pumps.
17. The device of any preceding embodiment, wherein the plurality of pumps comprises at least one diaphragm pump.
18. The device of any preceding embodiment, wherein the device comprises a comprises a valve for releasing and/or maintaining positive pressure, optionally a solenoid valve.
19. The device of any preceding embodiment, wherein:
   i) the sample module further comprises a regeneration unit configured to regenerate the matrix in any receptacle of the plurality of receptacles,
   ii) the multi-channel module further comprises a plurality of reservoirs for storing reagents for regeneration of the matrix,
   iii) the plurality of pumps is further configured to deliver the reagents for regeneration of the matrix to and/or through the matrix of any receptacle of the plurality of samples, and wherein:
   the multi-channel pump module and the sample module are further connected to enable regeneration of any matrix of the plurality of receptacles, and
   the means for housing the plurality of receptacles is further configured such that any of the plurality of the receptacles can be positioned for delivery of the reagents from the regeneration unit.
20. The device of embodiment 19, wherein the regeneration unit comprises a plurality of tubes configured to deliver the reagents to the matrix.
21. The device of embodiment 20, wherein the regeneration unit comprises a nozzle configured to force reagents through the matrix, preferably wherein the nozzle can form an airtight seal with any receptacle of the plurality of receptacles.
22. The device of any of embodiments 19 to 21, wherein the reagents for regeneration of the matrix comprise sodium hypochlorite, preferably 0.7% sodium hypochlorite.
23. A process for automated, matrix-based extraction of nucleic acid samples from a plurality of samples, comprising the steps of:
   i) receiving a sample of the plurality of samples in a receptacle of a plurality of receptacles,
   ii) binding a nucleic acid to a matrix,
   iii) washing the matrix to remove residual reagent, and
   iv) eluting the nucleic acid from the matrix;
   wherein a volume of sample used in the process and/or a volume of any reagent of a plurality of reagents used in the process are controlled using image processing; and
   wherein image processing is used to measure the volume of the sample and/or the volume of any reagent of a plurality of reagents received in the receptacle.
24. The process of embodiment 23, wherein the control of the volume of the sample and or the control of the volume of the reagent is performed continuously, preferably using closed loop feedback control.
25. The process of embodiment 23 or embodiment 24, wherein a positive pressure is used to force the sample and/or the reagent through the matrix.
26. The process of embodiment 25, wherein the positive air pressure is generated by a syringe pump or a diaphragm pump, preferably by a diaphragm pump.
27. The process of any of embodiments 23 to 26, wherein a backpressure measurement is used to determine the absence of residual reagent on the matrix used in the process.
28. The process of embodiment 27, wherein the backpressure measurement is performed in the receptacle.
29. The process of any of embodiments 23 to 28, wherein the matrix is a silica membrane, a glass fibre matrix or a zeolite.
30. The process of embodiment 29, wherein the matrix is a silica membrane comprised by a column, preferably a minispin column.
31. A device for automated extraction of nucleic acid samples from a plurality of samples, comprising:
   i) a sample module comprising:
      a) a plurality of receptacles for receiving the plurality of samples, wherein each receptacle of the plurality of receptacles comprises a matrix for nucleic acid binding,
      b) means for housing the plurality of receptacles,
      c) an extraction unit configured to extract the nucleic acid samples from any sample of the plurality of samples, and
      d) a regeneration unit configured to regenerate the matrix in any receptacle of the plurality of receptacles; and
   ii) a multi-channel pump module, comprising:
      e) a plurality of reservoirs for storing reagents for extraction of nucleic acid samples and a plurality of reservoirs for storing reagents for regeneration of the matrix, and
      f) a plurality of pumps configured to deliver the plurality of samples, the reagents for extraction of nucleic acid samples and/or the reagents for regeneration of the matrix to and/or through the matrix of any receptacle of the plurality of receptacles; and wherein:
         the multi-channel pump module and the sample module are connected such that the reagents can be delivered to and/or through any matrix of the plurality of receptacles to enable the extraction of the nucleic acid samples from the plurality of samples and such that any matrix of the plurality of receptacles can be regenerated, and
         the means for housing the plurality of receptacles is configured such that any of the plurality of the receptacles can be positioned for delivery of the reagents from the extraction unit or the regeneration unit.
32. The device of embodiment 31, wherein the extraction unit and the regeneration unit each comprise a plurality of tubes configured to deliver the sample and/or the reagents to the matrix.
33. The device of any of embodiments 31 or embodiment 32, wherein the extraction unit and the regeneration unit each comprise a nozzle configured to force the sample and/or the reagents through the matrix.
34. The device of embodiment 33, wherein the nozzle can form an airtight seal with any receptacle of the plurality of receptacles.
35. The device of embodiment any of embodiments 31 to 34, wherein the reagents for regeneration of the matrix comprise sodium hypochlorite, preferably 0.7% sodium hypochlorite.
36. The device of any of embodiments 31 to 35, wherein the device comprises an in-line lysis module for lysis of any sample of the plurality of samples.
37. The device of embodiment 36, wherein the in-line lysis module comprises a mechanical vibration device for mixing of the sample with a lysis buffer.
38. The device of any of embodiments 31 to 37, wherein the nucleic acid is DNA or RNA.
39. The device of any of embodiments 31 to 38, wherein the matrix is a silica membrane, a glass fibre matrix or a zeolite.
40. The device of any of embodiments 31 to 39, wherein the plurality of receptacles is a plurality of columns, preferably a plurality of mini spin columns.
41. The device of embodiment 40, wherein the plurality of receptacles is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 receptacles.
42. The device of any of embodiments 31 to 41, wherein the means for housing the plurality of receptacles is a carousel.
43. The device of any of embodiments 31 to 42, wherein the plurality of pumps comprises peristaltic pumps.
44. The device of any of embodiments 31 to 43, wherein the plurality of pumps comprises syringe pumps.
45. The device of any of embodiments 31 to 44, wherein the plurality of pumps comprises at least one diaphragm pump.
46. The device of any of embodiments 31 to 45, wherein the device comprises a valve for releasing and/or maintaining positive pressure, optionally a solenoid valve.
47. The device of any of embodiments 31 to 46, wherein the nozzle of the extraction unit and the nozzle of the regeneration unit are further configured to measure the air pressure in the receptacle.
48. The device of any of embodiments 31 to 47, wherein the device comprises means for measuring the volume of analyte in any receptacle of the plurality of receptacles.
49. The device of embodiment 48, wherein the volume measurements are used to control the plurality of pumps, preferably using closed loop feedback control.
50. The device of embodiment 47 and/or embodiment 48, wherein the pressure measurement is used together with the volume measurements to control the plurality of pumps.
51. The device of any of embodiments 48 to 50, wherein the means for measuring the volume of analyte is a camera system.
52. A process for automated, matrix-based extraction of nucleic acid samples from a plurality of samples using a plurality of matrices for nucleic acid binding, the process comprising a nucleic acid extraction step and a matrix regeneration step, wherein the nucleic acid extraction step and the matrix regeneration step are performed in-parallel.
53. The process of embodiment 52, wherein the regeneration step comprises contacting the matrix with a regeneration reagent, followed by removal of the regeneration reagent by washing the matrix with water.
54. The process of embodiment 53, wherein the removal of the regeneration reagent from the matrix is confirmed using a backpressure measurement.
55. The process of any of embodiments 52 to 54, wherein the matrix is a silica membrane silica membrane, a glass fibre matrix or a zeolite.
56. The process of embodiment 55, wherein the matrix is a silica membrane is comprised by a column, preferably a minispin column.
57. The process of any of embodiments 53-56, wherein the regeneration reagent is sodium hypochlorite, preferably 0.7% sodium hypochlorite
58. The process of any of embodiments 52 to 57, wherein the plurality of matrices is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 matrices.
59. The process of any of embodiments 52 to 58, wherein each matrix is regenerated at least 1, at least 2, at least 3, at least, 4, at least 5, at least 6, at least 7, at least 8, at least 9 or at least 10 times.

What is claimed is:

1. A device for automated extraction of nucleic acid samples from a plurality of samples, comprising:
i) a sample module comprising:
a) a plurality of receptacles for receiving the plurality of samples, wherein each receptacle of the plurality of receptacles comprises a matrix for nucleic acid binding,
b) means for housing the plurality of receptacles,
c) an extraction unit configured to extract the nucleic acid samples from any sample of the plurality of samples, and
d) a regeneration unit configured to regenerate the matrix in any receptacle of the plurality of receptacles; and
ii) a multi-channel pump module, comprising:
e) a plurality of reservoirs for storing reagents for extraction of nucleic acid samples and a plurality of reservoirs for storing reagents for regeneration of the matrix, and
f) at least one pump configured to deliver the plurality of samples, the reagents for extraction of nucleic acid samples and/or the reagents for regeneration of the matrix to and/or through the matrix of any receptacle of the plurality of receptacles; and wherein:
the multi-channel pump module and the sample module are connected such that the reagents can be delivered to and/or through any matrix of the plurality of receptacles to enable the extraction of the nucleic acid samples from the plurality of samples and such that any matrix of the plurality of receptacles can be regenerated, and
the means for housing the plurality of receptacles is configured such that any of the plurality of the receptacles can be positioned for delivery of the reagents from the extraction unit or the regeneration unit.

2. The device of claim 1, wherein the extraction unit and the regeneration unit each comprise a plurality of tubes configured to deliver the sample and/or the reagents to the matrix.

3. The device of any of claims 1 or claim 2, wherein the extraction unit and the regeneration unit each comprise a nozzle configured to force the sample and/or the reagents through the matrix.

4. The device of claim 3, wherein the nozzle can form an airtight seal with any receptacle of the plurality of receptacles.

5. The device of claim any of claims 1 to 4, wherein the reagents for regeneration of the matrix comprise sodium hypochlorite, preferably 0.7% sodium hypochlorite.

6. The device of any of claims 1 to 5, wherein the device comprises an in-line lysis module for lysis of any sample of the plurality of samples.

7. The device of claim 6, wherein the in-line lysis module comprises a mechanical vibration device for mixing of the sample with a lysis buffer.

8. The device of any of claims 1 to 7, wherein the nucleic acid is DNA or RNA.

9. The device of any of claims 1 to 8, wherein the matrix is a silica membrane, a glass fibre matrix or a zeolite.

10. The device of any of claims 1 to 9, wherein the plurality of receptacles is a plurality of columns, preferably a plurality of mini spin columns.

11. The device of claim 10, wherein the plurality of receptacles is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 receptacles.

12. The device of any of claims 1 to 11, wherein the means for housing the plurality of receptacles is a carousel.

13. The device of any of claims 1 to 12, wherein the at least one pump comprises peristaltic pumps.

14. The device of any of claims 1 to 13, wherein the at least one pump comprises syringe pumps.

15. The device of any of claims 1 to 14, wherein the at least one pump comprises at least one diaphragm pump.

16. The device of any of claims 1 to 15, wherein the device comprises a valve for releasing and/or maintaining positive pressure, optionally a solenoid valve.

17. The device of any of claims 1 to 16, wherein the nozzle of the extraction unit and the nozzle of the regeneration unit are further configured to measure the air pressure in the receptacle.

18. The device of any of claims 1 to 17, wherein the device comprises means for measuring the volume of analyte in any receptacle of the plurality of receptacles.

19. The device of claim 18, wherein the volume measurements are used to control the at least one pump, preferably using closed loop feedback control.

20. The device of claim 18 or claim 19, wherein the pressure measurement is used together with the volume measurements to control the at least one pump.

21. The device of any of claims 18 to 20, wherein the means for measuring the volume of analyte is a camera system.

22. The device of any of the preceding claims, wherein the at least one pump is a plurality of pumps.

23. The device of any of the preceding claims, wherein the device further comprises a suction system comprising a suction pump, a waste reservoir for collection of waste liquid and a linear actuator, the linear actuator configured to dock the waste reservoir of the suction system to the sample receptacle, optionally using a funnel.

24. A process for automated, matrix-based extraction of nucleic acid samples from a plurality of samples using a plurality of matrices for nucleic acid binding, the process comprising a nucleic acid extraction step and a matrix regeneration step, wherein the nucleic acid extraction step and the matrix regeneration step are performed in-parallel.

25. The process of claim 24, wherein the regeneration step comprises contacting the matrix with a regeneration reagent, followed by removal of the regeneration reagent by washing the matrix with water.

26. The process of claim 25, wherein the removal of the regeneration reagent from the matrix is confirmed using a backpressure measurement.

27. The process of any of claims 24 to 26, wherein the matrix is a silica membrane silica membrane, a glass fibre matrix or a zeolite.

28. The process of claim 27, wherein the matrix is a silica membrane is comprised by a column, preferably a minispin column.

29. The process of any of claims 25-28, wherein the regeneration reagent is sodium hypochlorite, preferably 0.7% sodium hypochlorite

30. The process of any of claims 24 to 29, wherein the plurality of matrices is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 matrices.

31. The process of any of claims 24 to 30, wherein each matrix is regenerated at least 1, at least 2, at least 3, at least, 4, at least 5, at least 6, at least 7, at least 8, at least 9 or at least 10 times.

32. A device for automated extraction of nucleic acid samples from a plurality of samples, comprising:
i) a sample module comprising:
a) a plurality of receptacles for receiving the plurality of samples, wherein each receptacle of the plurality of receptacles comprises a matrix for nucleic acid binding,
b) means for housing the plurality of receptacles, and
c) an extraction unit configured to extract the nucleic acid samples from any sample of the plurality of samples; and
ii) a multi-channel pump module, comprising:
d) a plurality of reservoirs for storing reagents for extraction of nucleic acid samples, and
e) a at least one pump configured to deliver the plurality of samples and/or the reagents for extraction of nucleic acid samples to and/or through the matrix of any receptacle of the plurality of receptacles; and wherein:
the device comprises means for measuring the volume of analyte in any receptacle of the plurality of receptacles,
the multi-channel pump module and the sample module are connected such that the reagents can be delivered to and/or through any matrix of the plurality of receptacles to enable the extraction of the nucleic acid samples from the plurality of samples, and
the means for housing the plurality of receptacles is configured such that any of the plurality of the receptacles can be positioned for delivery of the reagents from the extraction unit.

33. The device of claim 32, wherein the volume measurements are used to control the at least one pump, preferably using closed loop feedback control.

34. The device of claim 32 or claim 33, wherein the means for measuring the volume of analyte is a camera system.

35. The device of any one of claims 32 to 34, wherein the extraction unit comprises a nozzle configured to force the sample and/or the reagents through the matrix.

36. The device of claim 35, wherein the nozzle can form an airtight seal with any receptacle of the plurality of receptacles.

37. The device of claim 35 or claim 36, wherein the nozzle is configured to measure the air pressure in the receptacle.

38. The device of claim 37, wherein the pressure measurement is used together with the volume measurements to control the at least one pump.

39. The device of any one of claims 32 to 38, wherein the device comprises an in-line lysis module for lysis of any sample of the plurality of samples.

40. The device of claim 39, wherein the in-line lysis module comprises a mechanical vibration device for mixing of the sample with a lysis buffer.

41. The device of any one of claims 32 to 40, wherein the nucleic acid is DNA or RNA.

42. The device of any one of claims 32 to 41, wherein the matrix is a silica membrane, a glass fibre matrix or a zeolite.

43. The device of any one of claims 32 to 42, wherein the plurality of receptacles is a plurality of columns, preferably a plurality of mini spin columns.

44. The device of any one of claims 32 to 43, wherein the plurality of receptacles is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 receptacles.

45. The device of any one of claims 32 to 44, wherein the means for housing the plurality of receptacles is a carousel.

46. The device of any one of claims 32 to 45, wherein the at least one pump comprises peristaltic pumps.

47. The device of any one of claims 32 to 46, wherein the at least one pump comprises syringe pumps.

48. The device of any one of claims 32 to 47, wherein the at least one pump comprises at least one diaphragm pump.

49. The device of any one of claims 32 to 48, wherein the device comprises a comprises a valve for releasing and/or maintaining positive pressure, optionally a solenoid valve.

50. The device of any one of claims 32 to 49, wherein:
i) the sample module further comprises a regeneration unit configured to regenerate the matrix in any receptacle of the plurality of receptacles,
ii) the multi-channel module further comprises a plurality of reservoirs for storing reagents for regeneration of the matrix,
iii) the at least one pump is further configured to deliver the reagents for regeneration of the matrix to and/or through the matrix of any receptacle of the plurality of samples, and wherein:
the multi-channel pump module and the sample module are further connected to enable regeneration of any matrix of the plurality of receptacles, and
the means for housing the plurality of receptacles is further configured such that any of the plurality of the receptacles can be positioned for delivery of the reagents from the regeneration unit.

51. The device of claim 50, wherein the regeneration unit comprises a plurality of tubes configured to deliver the reagents to the matrix.

52. The device of claim 51, wherein the regeneration unit comprises a nozzle configured to force reagents through the matrix, preferably wherein the nozzle can form an airtight seal with any receptacle of the plurality of receptacles.

53. The device of any of claims 50 to 52, wherein the reagents for regeneration of the matrix comprise sodium hypochlorite, preferably 0.7% sodium hypochlorite.

54. The device of any one of claims 32 to 53, wherein the at least one pump is a plurality of pumps.

55. The device of any of claims 32 to 54 preceding claims, wherein the device further comprises a suction system comprising a suction pump, a waste reservoir for collection of waste liquid and a linear actuator, the linear actuator configured to dock the waste reservoir of the suction system to the sample receptacle, optionally using a funnel.

56. A process for automated, matrix-based extraction of nucleic acid samples from a plurality of samples, comprising the steps of:
i) receiving a sample of the plurality of samples in a receptacle of a plurality of receptacles,
ii) binding a nucleic acid to a matrix,
iii) washing the matrix to remove residual reagent, and
iv) eluting the nucleic acid from the matrix;
wherein a volume of sample used in the process and/or a volume of any reagent of a plurality of reagents used in the process are controlled using image processing; and
wherein image processing is used to measure the volume of the sample and/or the volume of any reagent of a plurality of reagents received in the receptacle.

57. The process of claim 56, wherein the control of the volume of the sample and or the control of the volume of the reagent is performed continuously, preferably using closed loop feedback control.

58. The process of claim 56 or claim 57, wherein a positive pressure is used to force the sample and/or the reagent through the matrix.

59. The process of claim 58, wherein the positive air pressure is generated by a syringe pump or a diaphragm pump, preferably by a diaphragm pump.

60. The process of any of claims 56 to 59, wherein a backpressure measurement is used to determine the absence of residual reagent on the matrix used in the process.

61. The process of claim 60, wherein the backpressure measurement is performed in the receptacle.

62. The process of any of claims 56 to 61, wherein the matrix is a silica membrane, a glass fibre matrix or a zeolite.

63. The process of claim 62, wherein the matrix is a silica membrane comprised by a column, preferably a minispin column.
